(19) United States
(12) Patent Application Publication
(10) Pub. No.: US 2009/0099448 A1
(43) Pub. Date: Apr. 16, 2009

(54) Human or animal organ imaging system which can be used to measure the elasticity of the organ

(75) Inventors: Laurent Sandrin, I'Hay-les-Roses (FR); Jean-Michel Hasquenoph, Couilly-Pont-aux-Dames (FR)

Correspondence Address: FLETCHER YODER;Patrick S. Yoder, P.O. Box 692289, Houston, TX, 77269-2289, US

(73) Assignee: ECHOSENS, Paris (FR)

(21) Appl. No.: 12/069,286

(22) Filed: Feb. 8, 2008

(30) Foreign Application Priority Data
Aug. 12, 2005 (FR) 05/52503
Aug. 3, 2006 (FR) PCT/FR2006/001891

(51) Int. Cl. A61B 8/00

(52) U.S. Cl. 600/438

(57) ABSTRACT

A system and method are provided for an imaging system that may be used to measure the elasticity of an organ. In the system, an electronic transducer forms an image of a human or animal organ. A mobile part may be moved in translation or rotation, and is arranged to induce the propagation of low frequency vibration in the direction of the organ when actuated. The mobile part delivers the vibrational energy against the human or animal body. The transducer has a fixed part that also emits similar energy. By application of energy to the mobile and fixed parts, and by collection of energy, a control and calculation module may compute parameters of internal organs, such as elasticity.

The invention relates to an ultrasonic transducer which is used to form an image of a human or animal organ and which can also be used to measure the elasticity of said organ as well as a human or animal organ imaging system comprising such a transducer.

Measuring the elasticity of a human or animal organ using a low frequency vibration propagating in the human or animal body in the direction of such organ is known. The vibration is created by a low frequency pulse created for instance by an ultrasonic transducer. The transducer makes it possible to examine the propagation of the vibration and to form an image of the organ the elasticity of which is measured by ultrasonic illumination. Such a device is for instance described in document FR-2 843 290.

Such document teaches that the ultrasonic transducer creating the low frequency vibration can have small dimensions, so as to allow the device to be positioned in reduced size spaces, such as the intercostal space, if the elasticity of the liver for instance is to be measured. However, in order to obtain an ultrasonic image with a correct resolution, the transducer should have a large size, since the ultrasonic focal spot is inversely proportional to such size.

Document FR-2 843 290 also teaches that the low frequency pulse can be generated by an array of ultrasonic elements which is actuated. However, as the array of ultrasonic elements has a very large size, the ultrasonic image has a correct resolution, but the low frequency vibration undergoes diffraction effects and the array of ultrasonic elements cannot be introduced into a small size space.

The object of the invention is to remedy such disadvantages by providing a transducer, the dimension of which is adapted to obtain an image with a correct resolution, and a small area part of which at least is movable in order to create an ultrasonic vibration which can further be used to measure the elasticity of such organ while avoiding the diffraction effects and which can be introduced into a small size space.

The ultrasonic transducer is used in an imaging system. In such an embodiment, delays and amplitudes calculation problems arise, since the motion of the mobile part with respect to the fixed parts which entails a delay in the emission and the reception of ultrasounds should be taken into account. The invention provides a system, the analyzing means of which make it possible to take such motion into account.

For this purpose and according to a first aspect, the invention relates to an ultrasonic transducer which is used to form an image of a human or animal organ and which is also used to measure the elasticity of said organ, said transducer comprising at least one mobile part 3 arranged to induce the propagation of a low frequency vibration in the direction of the organ when the mobile part 3 is actuated and delivers an impact against a human or animal body, the transducer 2 further comprising at least one fixed part 4.

Thus, the invention makes it possible to take into account the constraints related to the small dimension that the surface creating the low frequency vibration must have, and to the big size of the ultrasonic transducer which makes it possible to obtain an ultrasonic image with a correct resolution.

According to a second aspect, the invention relates to a human or animal organ imaging system also used to measure the elasticity of said organ, comprising a transducer such as described hereabove, said system further comprising a control and calculation device arranged to control the emission and the reception of ultrasounds and the motion of the mobile part, said device comprising means for analysing the ultrasounds emitted and received by the transducer and the motion of the mobile part, in order to create the image of the organ and said analysing means further making it possible to analyse the low frequency vibration induced in order to measure the elasticity of said organ.

According to one embodiment, the system comprises a position sensor for the mobile part. Thus, the analysing means comprise adjustment means for the delays upon the emission and the reception of ultrasonic signals emitted and received by the mobile part when said part moves in order to make such signals coincide with the signals emitted and received by the fixed part, said means using the information supplied by the position sensor in real time. Thus, a coincidence between the signals emitted and received by the mobile part and those received by the fixed part is ensured and the motion of the mobile part is taken into account for creating the image of the human or animal organ.

Other aspects and advantages of the invention will become apparent by reading the following description, and referring to the appended drawings.

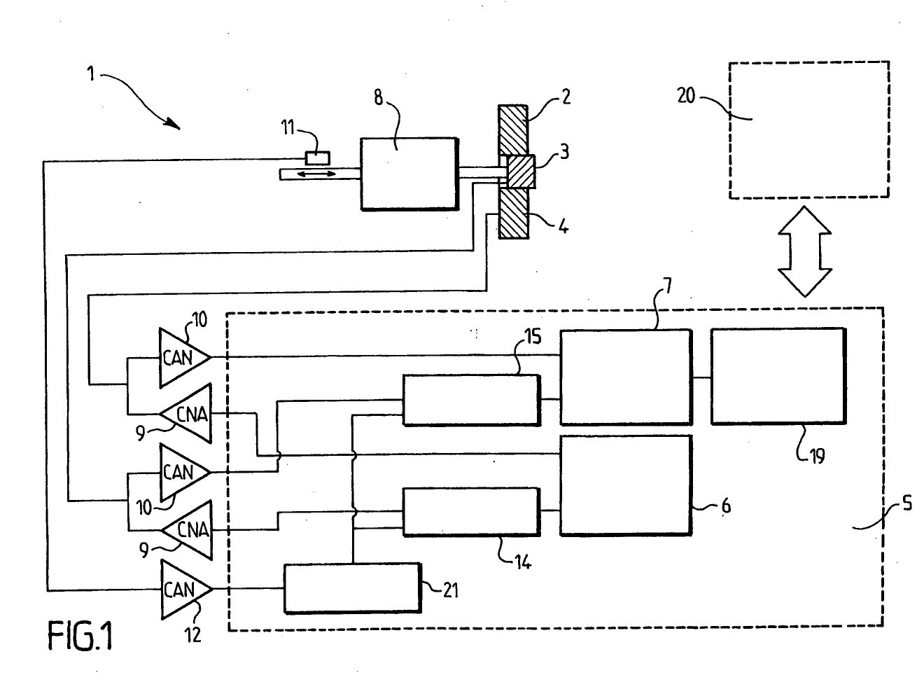

FIG. 1 shows schematically an imaging system according to the invention.

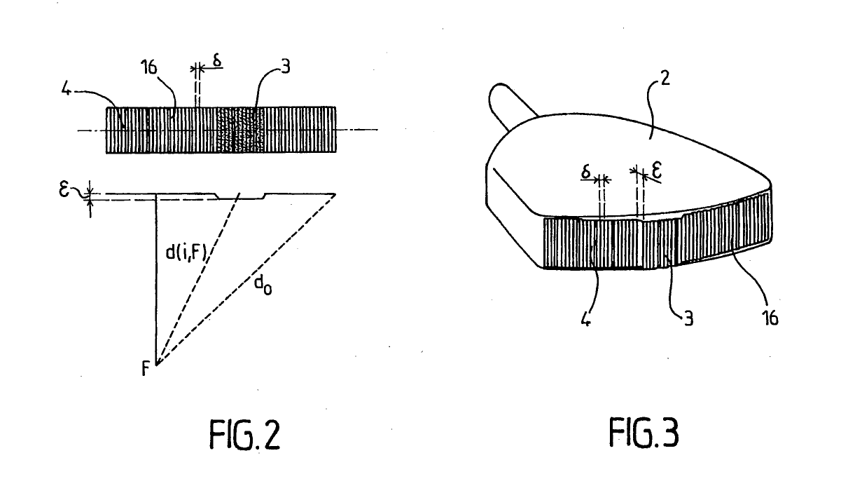

FIG. 2 schematically shows the ultrasonic transducer of FIG. 1, illustrating the motion which can be made by the mobile part.

FIG. 3 schematically shows in perspective the ultrasonic transducer according to the invention.

Referring to FIG. 1, a human or animal organ imaging system 1 is described, which is also used for measuring the elasticity of said organ. The system 1 comprises an ultrasonic transducer 2 having a general size adapted for obtaining an ultrasonic image with a correct resolution. For this purpose, the transducer has for instance the dimension of a standard array of ultrasonic elements.

The transducer 2 comprises at least one mobile part 3 and at least one fixed part 4. According to the embodiment shown in the Figures, the mobile part 3 moves in translation according to a direction which is substantially perpendicular to the direction in which the transducer 2 extends. According to other embodiments which are not shown, the mobile part 3 can move in translation and along the direction in which the transducer extends or moves in rotation, so as to create the low frequency vibration.

The dimension of the mobile part 3 is reduced with respect to the dimension of the transducer 2. More particularly, the mobile part 3 has a surface which is adapted to limit the diffraction effects upon the propagation of the low frequency vibration and so that the mobile part can be introduced into the intercostal space of the human or animal body. The mobile part is for instance substantially placed at the centre of the transducer 2 between two fixed parts 4, as shown in the FIGS. 1 and 3.

According to an embodiment, not shown, the transducer 2 can include a plurality of mobile parts 3 alternately distributed with fixed parts 4 along the transducer 2. According to another embodiment, the transducer 2 can include mobile parts 3 positioned adjacent to one another.

The system 1 comprises means 8 for actuating the mobile part 3. Thus, the mobile part is arranged to induce the propagation of a low frequency vibration in the direction of the organ, when the mobile part 3 is actuated in translation and delivers an impact against the human or animal body. The actuation means 8 are controlled by a control and calculation device 5 which further controls the emission of ultrasounds by the fixed part 4 and the mobile part 3. In the case where several mobile parts 3 are provided, the control and calculation device 5 can control the actuation means 8, so that the mobile parts 3 move in opposite phase.

The control and calculation device 5 includes an emission signal generation module 6 and a track formation module 7. The generation module 6 provides an ultrasonic emission signal to the mobile part 3 and to the fixed part 4 using an emission law selected to form an image with a correct resolution of the examined organ. The generation module 6 is connected to the fixed part 4 and to the mobile part 3 through digital/analog converters 9 as shown in FIG. 1. Similarly, the fixed part 4 and the mobile part 3 are connected to the track formation module 7 through analog/digital converters 10. The track formation module 7 can then be connected to an elasticity calculation module 19. The control and calculation module 5 is connected to a display device 20 which makes it possible to display the image of the organ and the results of the measurement of the elasticity of the organ or an operation system, an interface with the user for example.

A position sensor 11 for the mobile part 3 is associated with such mobile part 3. The position sensor 11 makes it possible to know the position of the mobile part 3 when the latter moves and is no longer aligned with the fixed part 4. The motion of the mobile part 3 can occur on a distance ε as shown in FIG. 2. The position sensor 11 is, for example, a Hall effect sensor connected to the generation module 6 and to the track formation module 7 through an analog/digital converter 12 capable of digitising the signal supplied by such sensor. On the other hand, the control and calculation device 5 includes ultrasounds generation and processing means for the ultrasounds emitted and received by the transducer so as to create the image of the organ and said generation and processing means further making it possible to analyse the low frequency vibration induced in order to measure the elasticity of said organ. Such generation and processing means 6 and 7 are associated with delay adjustment modules in real time 14 and 15, upon the emission and the reception of the ultrasonic signals emitted and received by the mobile part when said part moves, in order to make such signals coincide with the signals emitted and received by the fixed part. They further include gain adjustment modules in real time 14 and 15, upon the emission of ultrasonic signals emitted by the mobile part, when such part moves in order to make such gains coincide with the ultrasonic signals gains emitted by the fixed parts. Such adjustments modules 14 and 15 are positioned between a correction calculation module 21 and the generation module 6 and the track formation module 7, as shown in FIG. 1, and their operation is described hereinunder.

The position sensor 11 delivers a signal which is representative of the motion ε of the mobile part 3. Such signal is digitised by means of an analog/digital converter 12 then transmitted to the correction calculation module 21 which uses, in real time, the information supplied by the positioned sensor 11. The corrections are supplied to the adjustment modules 14 and 15. The ultrasonic transducer 2 is a conventional transducer which comprises a plurality of elements 16 which can emit and receive ultrasounds. δ indicates the pitch between each element 16 which means the distance separating two consecutive elements. Besides, F indicates the focal spot located on the line formed by the transducer 2. d(i, F) is the distance between the focal spot F and one element i of the transducer 2 and dₒ is the greatest distance between an element 16 of the opening used and the focal spot F. Vs is the speed of the ultrasounds propagating in the body.

According to the indications above, the delays upon the emissions Rₑ and the delays upon the reception Rᵣ for a stationary element i and for the construction of a line identified as c can be calculated as follows:

$$\{ \begin{matrix}{R}_{e} ( c , i ) = \frac{{d}_{0} - d ( i , F )}{{V}_{s}} \\ {R}_{r} ( c , i ) = \frac{d ( i , F )}{{V}_{s}}\end{matrix}$$

For an element i shifted by a distance ε with respect to the position in which it is aligned with the elements 16 of the fixed part, the corrected delays can be calculated as follows:

$$\{ \begin{matrix}{R}_{e} ( c , i ) = \frac{{d}_{0} - \sqrt{{[ F - ɛ ]}^{2} + {[ ( i - c ) δ ]}^{2}}}{{V}_{s}} \\ {R}_{r} ( c , i ) = \frac{\sqrt{{[ F - ɛ ]}^{2} + {[ ( i - c ) δ ]}^{2}}}{{V}_{s}}\end{matrix}$$

The corrections with respect to the delays applied when the motion ε is null, can be noted as follows when the motion ε is negligible with respect to the focal length.

$$\{ \begin{matrix}Δ {R}_{e} ( c , i ) = \frac{ɛ}{{V}_{s}} {( 1 + \frac{{[ ( i - c ) δ ]}^{2}}{{F}^{2}} )}^{- 1 / 2} \\ Δ {R}_{r} ( c , i ) = Δ {R}_{e} ( c , i )\end{matrix}$$

The correction calculation module 21 makes it possible to perform such calculations and to provide the results to the adjustment modules upon the emission and the reception 14 and 15 as shown in FIG. 1. In the track formation module 7, a summer makes it possible to add the signals received by the fixed part 4 and those received by the mobile part 3 in order to create the image of the organ.

The data supplied by the position sensor 11 are used in real time in order to image the organ at any time without any significant processing delay.

The adjustment modules 14 and 15 also make it possible to adapt the gain to the emission of the ultrasonic signals emitted by the mobile part 3 as a function of its position, as shown in FIG. 1. As a matter of fact, since the mobile part 3 does not emit signals from the same place as the fixed part 4, it is necessary to adapt the amplitude of the signals emitted by the mobile part 3 so that it is equal to that of the signals emitted by the fixed part 4.

The measure of the elasticity of the organ using the system 1 is known in itself and can for instance originate from the solution provided by document FR-2 843 290.

1. An ultrasonic transducer used to form the image of a human or animal organ further which can further be used to measure the elasticity of said organ, comprising at least one mobile part arranged to induce the propagation of a low frequency vibration in the direction of the organ when the mobile part is actuated and delivers an impact against the human or animal body, the transducer further comprising at least one fixed part.

2. A transducer according to claim 1, comprising means for actuating the mobile part.

3. A transducer according to claim 1, wherein the mobile part is movable in translation.

4. A transducer according to claim 1, wherein the mobile part is movable in rotation.

5. A transducer according to claim 1, wherein the mobile part has a surface adapted to limit the diffraction effects upon the propagation of the low frequency vibration and so that the mobile part can be introduced into the intercostal space of the human or animal body.

6. A transducer according to claim 1, comprising a plurality of mobile parts and a plurality of fixed parts.

7. An imaging system for a human or animal organ which can further be used to measure the elasticity of said organ, according to claim 1, said system further comprising a control and calculation device arranged to control the emission and the reception of ultrasounds and the motion of the mobile part, said device comprising ultrasound generation and processing means for the ultrasounds emitted and received by the transducer and the motion of the mobile part so as to create the image of the organ.

8. A system according to the claim 7, comprising a position sensor for the mobile part.

9. A system according to claim 8, wherein the position sensor is a Hall effect sensor, the control and calculation device comprising an analog/digital converter capable of digitising the signal supplied by said sensor.

10. A system according to claim 8, wherein the generation and processing means comprise delays adjustment modules for adjusting delays of the emission and the reception of ultrasound signals emitted and received by the mobile part when said part moves so as to make such signals coincide with the signals emitted and received by the fixed part, said modules using the information supplied by the position sensor in real time, through the corrections calculation module.

11. A system according to claim 8, wherein the generation and processing means comprise gains adjustment modules for adjusting gains of the emission of ultrasound signals emitted by the mobile part, when said part moves so as to make such gains coincide with the gains of the ultrasound signals emitted by the fixed part, said modules using the information supplied by the position sensor in real time, through the corrections calculation module.
